(19) United States
(12) Patent Application Publication
(10) Pub. No.: US 2011/0293528 A1
(43) Pub. Date: Dec. 1, 2011

(54) ENEMA PREPARATIONS AND THEIR USE

(75) Inventors: Aslak Godal, Oslo (NO); Jo Klaveness, Oslo (NO)

(73) Assignee: PHOTOCURE ASA, Oslo (NO)

(21) Appl. No.: 13/141,919

(22) PCT Filed: Dec. 23, 2009
(86) PCT No.: PCT/EP2009/009293

§ 371 Date: Aug. 17, 2011

(30) Foreign Application Priority Data
Dec. 23, 2008 (GB) 0823472.6

(51) Int. Cl. A61K 49/06; A61K 31/221; A61K 49/04; A61P 1/00; A61P 1/04; A61P 31/00; A61K 31/197; A61P 35/00

(52) U.S. Cl. 424/9.3; 514/561; 514/551; 424/9.4

(57) ABSTRACT

This invention relates to improved methods of photodynamic treatment and diagnosis of cancer and non-cancerous conditions, and in particular to improved enema preparations for use in such methods, said enema preparations comprising a photosensitiser which is 5-aminolevulinic acid (5-ALA) or a precursor or derivative thereof, e.g. a 5-ALA ester. Such preparations may further comprise one or more viscosity enhancing agents, mucoadhesive or mucolytic agents, penetration enhancers or chelating agents. The methods and preparations herein described are particularly suitable for use in photodynamic methods of treating and/or diagnosing cancer and non-cancerous conditions in the colon and/or rectum.

This invention relates to improved methods of photodynamic treatment and diagnosis of cancer and non-cancerous conditions, and in particular to improved enema preparations for use in such methods, said enema preparations comprising a photosensitiser which is 5-aminolevulinic acid (5-ALA) or a precursor or derivative thereof, e.g. a 5-ALA ester. The methods and preparations herein described are particularly suitable for use in photodynamic methods of treating and/or diagnosing cancer and non-cancerous conditions in the colon and/or rectum.

Photodynamic therapy (PDT) is a relatively new technique that has been used in the treatment of various cancers as well as other diseases. PDT involves the administration of photosensitizing agents followed by exposure to photoactivating light in order to activate the photosensitizing agents and convert them into cytotoxic form resulting in the destruction of cells and thus treatment of the disease. Several photosensitizing agents are known and described in the literature including 5-aminolevulinic acid (5-ALA) and certain derivatives thereof, e.g. 5-ALA esters.

Currently three pharmaceutical products comprising 5-ALA or an ester thereof are in clinical use for PDT and photodynamic diagnosis (PDD). These are Metvix® (Galderma, Switzerland), Hexvix® developed by Photocure ASA (Oslo, Norway) and Levulan Kerastick® developed by DUSA Pharmaceuticals (Canada). Metvix® is a dermal product for treatment of actinic keratosis and basal cell carcinoma which comprises methyl ALA ester in an emulsion (cream). Hexvix® is an aqueous solution which comprises hexyl ALA ester for instillation into the urine bladder for diagnosis of bladder cancer. Levulan Kerastick® is a 2-compartment formulation that is used to prepare a solution of 5-ALA immediately before application. This product can be used for the treatment of skin diseases.

An area of the body which is difficult to treat using conventional PDT or PDD methods is the lower part of the gastrointestinal tract, in particular the colon and rectum which may be associated with a number of serious and life-threatening diseases like colitis, colorectal cancer, Crohn's disease, irritable bowel disease and various local infections. Potentially the most serious of these is colorectal cancer. Current diagnostic methods for colorectal cancer include monitoring of clinical symptoms like blood in the stools, lower abdominal pain or weight loss, coloscopy and X-ray based imaging methods. The prognosis of patients with colorectal cancer depends, as with most other cancer forms, on disease stage at the time of diagnosis and especially on whether the patient has developed distant metastasis. There are several therapeutic drugs in clinical use today for treating colorectal cancer, however, current drugs have their clinical limitations and there remains a medical need for further therapeutic regimes and alternative methods of early diagnosis.

Conventional oral formulations comprising 5-ALA and derivatives thereof, such as solutions, suspensions, classical tablets and capsules containing aqueous formulations may have several disadvantages when used for the diagnosis and/or therapy of conditions in the lower part of the gastrointestinal system. These relate to shelf life stability of the pharmaceutical product, in vivo stability of the product during its passage through the whole gastrointestinal system, and systemic toxicity as a result of absorption of 5-ALA or derivatives thereof. Systemic absorption results in a reduction in clinical efficacy at the desired treatment site. Reduced efficacy is primarily a result of a non-homogenous and low concentration of 5-ALA or derivatives thereof in the lower part of the gastrointestinal system. In order for oral formulations to develop the desired clinical effects, it therefore becomes necessary for the amount of active ingredients to be increased. However, this can cause adverse reactions.

B. Mayinger et al. in Endoscopy 40: 106-109, 2008 describe a clinical study on detection of pre-malignant conditions in the colon by photodynamic diagnosis using enemas comprising 5-aminolevulinic acid hexyl ester and fluorescence endoscopy as a means of detection. The enemas used in the study comprise 200 mg 5-aminolevulinic acid hexyl ester (HAL) dissolved in 500 ml or 1000 ml sterile phosphate buffered saline. The final concentration of 5-aminolevulinic acid hexyl ester in these formulations is 1.6 and 0.8 mmol per litre, respectively. The authors show that the use of PDD detects 28% more polyps than when using white light endoscopic imaging. The best results were achieved with 500 ml of 1.6 mmol per litre 5-ALA hexyl ester which was instilled over 30 minutes and was retained for 60 minutes. The overall time prior to fluorescence detection was thus 90 minutes. A blocking balloon had to be used to prevent leakage of the enema during said 60 minutes retention time.

E. Endlicher et al. in Gastrointestinal Endoscopy 60(3): 449-454, 2004 have used a 5-ALA hexyl ester enema for the photodynamic detection of rectal adenoma or rectal cancer in patients. An instillation time of 30-45 minutes of 3.2 mmol per litre HAL, with a rest time of 30 minutes (overall time prior to detection: 60-75 minutes), gave satisfactory results.

H. Messmann et al. in Gut 52: 1003-1007, 2003 have used a 5-ALA enema for the photodynamic detection of low and high grade dysplasia in patients with ulcerative colitis. 3 g 5-ALA was dissolved in 250 ml 0.9% NaCl (the 5-ALA concentration corresponds to 72 mM) to prepare the enema which was instilled over 1 hour with a rest time of 1-2 hours resulting in an overall time before detection of 2-3 hours.

Despite the work done by Mayinger, Endlicher and Messmann, there is currently no product on the market for photodynamic diagnosis (PDD) or photodynamic therapy (PDT) of cancer or non-cancerous conditions, e.g. non-cancerous lesions in the colon or rectum. What is apparent from the studies carried out by Mayinger and Endlicher is that there is scope for improvement with regard to the enema procedure, i.e. instillation and incubation. For patients the length of the procedure is unpleasant with many patients experiencing leakage and difficulties in retaining the enema. For hospitals, which nowadays are required to economise and which in return are streamlining their procedures to achieve a high patient throughput, the enema procedure poses a challenge and, if reimbursement does not match expenses, such a procedure—despite resulting in an improved diagnosis—will hardly find acceptance. Private practices often do not have enough health personnel to carry out such procedures, which require that a nurse or physician attends to the patient during installation and incubation. Moreover, the patient needs to be monitored and turned to the side and back to ensure that the whole colon is covered by the enema.

A medical need thus remains for methods for earlier diagnosis of conditions in the lower gastrointestinal tract, e.g. the colon and rectum, especially for the diagnosis of colorectal cancer. In particular, a need exists for improved methods for the diagnosis of colorectal cancer and other non-cancer related conditions in the colon or rectum.

We have now developed improved methods of photodynamic therapy and photodynamic diagnosis in which a photosensitising agent comprising 5-ALA or a precursor or derivative thereof is administered in the form of an enema. Such methods have significantly improved therapeutic and diagnostic effects compared to conventional oral preparations and have the further advantage that a substantial volume of the agent can be administered directly to the afflicted area.

In particular we have developed such methods which require less time to perform compared to the methods described in the prior art. We have also developed novel enema formulations which have improved acceptability to patients, hospitals and private practices by shortening the overall time of the enema procedure.

Viewed from one aspect the invention thus provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in the photodynamic treatment or diagnosis of cancer or a non-cancerous condition in the lower part of the gastrointestinal tract.

In a further aspect the invention provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in the photodynamic therapy (PDT) of cancer in the lower part of the gastrointestinal tract, especially colorectal cancer.

In a yet further aspect the invention provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in the photodynamic therapy (PDT) of a non-cancerous condition, preferably such a condition which is not pre-malignant, in the lower part of the gastrointestinal system.

In a still further aspect the invention provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in the photodynamic diagnosis (PDD) of a non-cancerous condition in the lower part of the gastrointestinal system.

In another aspect the invention provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in the photodynamic diagnosis (PDD) of a cancerous condition in the lower part of the gastrointestinal system.

The diagnostic methods described herein may also be performed during surgery in which the enema preparation is given to the patient prior to surgery and surgery is then performed under blue light. The fact that the lesion or disease fluoresce under blue light aids the surgeon in defining the “surgical border” and thereby enables a more selective resection of the diseased area, e.g. a tumour. Use of the enema preparations herein described in methods of surgery forms a further aspect of the invention.

The therapeutic and diagnostic methods herein described may also be used in the form of a combined therapy. For example, a course of PDT performed in relation to a cancerous or non-cancerous condition using any of the methods herein described may be followed by a PDD method, e.g. to determine the extent to which PDT has been effective and/or to detect any re-occurrence of the condition. Also, a course of PDD performed in relation to a cancerous or non-cancerous condition using any of the methods herein described may be followed by a PDT method, e.g. to treat cancerous or non-cancerous conditions which have been detected by PDD.

In a further aspect the invention thus provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in a method which comprises the steps of:
   (i) conducting photodynamic treatment of cancer or a non-cancerous condition in the lower part of the gastrointestinal system, e.g. the colon or rectum, of a patient; and
   (ii) conducting photodynamic diagnosis on said patient.

At least one of steps (i) and (ii) is performed following administration to said patient of an enema preparation according to the invention. Preferably, steps (i) and (ii) will both be performed following administration of such an enema preparation.

In a further aspect the invention thus provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;
   for use in a method which comprises the steps of:
   (i) conducting photodynamic diagnosis of cancer or a non-cancerous condition in the lower part of the gastrointestinal system, e.g. the colon or rectum, of a patient; and
   (ii) conducting photodynamic treatment on said patient.

At least one of steps (i) and (ii) is performed following administration to said patient of an enema preparation according to the invention. Preferably, steps (i) and (ii) will both be performed following administration of such an enema preparation.

In a still further aspect the invention provides a method of photodynamic treatment or diagnosis of cancer or a non-cancerous condition in a patient, said method comprising the steps of:

(i) administering to said patient an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents;

(ii) optionally waiting for a time period for the photosensitiser to achieve an effective tissue concentration at the desired site; and

(iii) photoactivating the photosensitiser.

Prior to carrying out the therapeutic and diagnostic methods herein described it is preferred that the lower part of the gastrointestinal tract, e.g. the colon and rectum, should be evacuated, i.e. cleansed. This may be achieved in several ways conventionally known in the art, for example using a conventional enema procedure such as the use of an isotonic saline enema or the administration of laxative medications which may be taken orally. Typical products for cleansing include bisacodyl suppositories like Laxbene® (Merckle GmbH, Germany), oral formulations like Delcoprep® (DeltaSelect, Germany) and Endofalk® (DR.Falk GmbH, Germany), enemas comprising bisacodyl like Toilax® (Orion, Finland), rectal solutions containing sodium dioctylsulphosuccinate like Klyx (Ferring, Sweden) and enemas comprising sodium lauryl sulphate like Microlax® (McNeil, Sweden). Typically, the patient would also be required to fast, e.g. for a period of up to 12 hours prior to treatment.

More preferably, the invention thus provides a method of photodynamic treatment or diagnosis of cancer or a non-cancerous condition in a patient, said method comprising the steps of:
(i) evacuating the lower part of the gastrointestinal system of said patient;
(ii) optionally insufflating the lower part of the gastrointestinal system, e.g. with air or a gas;
(iii) administering to said patient an enema preparation which comprises a photosensitiser which is 5-ALA or a precursor or derivative thereof, e.g. an ALA ester, and at least one pharmaceutically acceptable carrier or excipient and which further comprises one or more of the following:
   a) one or more viscosity enhancing agents;
   b) one or more mucoadhesive agents or one or more mucolytic agents;
   c) one or more penetration enhancers; and
   d) one or more chelating agents;
(iv) optionally waiting for a time period necessary for the photosensitiser to achieve an effective tissue concentration at the desired site;
(v) optionally insufflating the lower part of the gastrointestinal system, e.g. with air or a gas; and
(vi) photoactivating the photosensitiser.

Following administration of the enema preparation, a balloon may be inserted into the opening of the rectum to avoid leakage of the product. To enhance homogenous filling of the whole colon the patient may be moved from one side to the other, and also requested to move their head a little up and down.

As used herein, the terms “cancer” and “cancerous” are used in connection with conditions where malignant cells are present. Pre-malignant conditions are thus not encompassed by these terms.

The term “non-cancerous” may include pre-malignant conditions. However, preferred non-cancerous conditions for treatment in accordance with the invention are those which are not pre-malignant.

As used herein the term “treatment” or “therapy” encompasses curative as well as prophylactic treatment or therapy.

The term “precursors” as used herein refers to precursors for 5-ALA which are converted metabolically to it and are thus essentially equivalent thereto. Thus the term “precursor” covers biological precursors for protoporphyrin in the metabolic pathway for haem biosynthesis. The term “derivatives” includes pharmaceutically acceptable salts and chemically modified agents, for example esters such as 5-ALA esters.

The use of 5-ALA and derivatives thereof, e.g. 5-ALA esters in PDT and PDD is well known in the scientific and patent literature (see, for example, WO 2006/051269, WO 2005/092838, WO 03/011265, WO 02/09690, WO 02/10120 and U.S. Pat. No. 6,034,267, the contents of which are incorporated herein by reference). All such derivatives of 5-ALA and their pharmaceutically acceptable salts are suitable for use in the methods herein described.

The 5-ALA derivatives useful in accordance with the invention may be any derivative of 5-ALA capable of forming protoporphyrin IX (PpIX) or any other photosensitiser, e.g. a PpIX derivative in vivo. Typically, such derivatives will be a precursor of PpIX or of a PpIX derivative, e.g. a PpIX ester, in the biosynthetic pathway for haem and which are therefore capable of inducing an accumulation of PpIX in vivo at the site of the administration. Suitable precursors of PpIX or PpIX derivatives include 5-ALA prodrugs which might be able to form 5-ALA in vivo as an intermediate in the biosynthesis of PpIX or which may be converted, e.g. enzymatically converted, to porphyrins without forming 5-ALA as an intermediate. 5-ALA esters and pharmaceutically acceptable salts thereof, are among the preferred photosensitisers for use in the methods herein described.

Esters of 5-aminolevulinic acid and N-substituted derivatives thereof are preferred photosensitisers for use in the invention. Those compounds in which the 5-amino group is unsubstituted, i.e. the ALA esters, are particularly preferred. Such compounds are generally known and described in the literature (see, for example, WO 96/28412 and WO 02/10120 to Photocure ASA, the contents of which are incorporated herein by reference).

Esters of 5-aminolevulinic acid with substituted or unsubstituted, preferably substituted, alkanols, i.e. alkyl esters and substituted alkyl esters, are especially preferred photosensitisers for use in the invention. Examples of such compounds include those of formula I:

R²₂N—CH₂COCH₂—CH₂CO—OR¹ (I)

wherein
R¹ represents a substituted or unsubstituted alkyl group; and
R² each independently represents a hydrogen atom or a group R¹.

As used herein, the term “alkyl”, unless stated otherwise, includes any long or short chain, cyclic, straight-chained or branched, saturated or unsaturated aliphatic hydrocarbon group. The unsaturated alkyl groups may be mono- or polyunsaturated and include both alkenyl and alkynyl groups. Unless stated otherwise, such alkyl groups may contain up to 40 carbon atoms. However, alkyl groups containing up to 30 carbon atoms, preferably up to 10, particularly preferably up to 8, especially preferably up to 6 carbon atoms are preferred.

In compounds of formula I, the R¹ groups are substituted or unsubstituted alkyl groups. If R¹ is a substituted alkyl group, one or more substituents are either attached to the alkyl group and/or interrupt the alkyl group. Suitable substituents that are attached to the alkyl group are those selected from: hydroxy, alkoxy, acyloxy, alkoxycarbonyloxy, amino, aryl, nitro, oxo, fluoro, —SR₃, —NR³₂ and —PR³₂, wherein R³ is a hydrogen atom or a C₁₋₆ alkyl group. Suitable substituents that interrupt the alkyl group are those selected from: —O—, —S— or —PR₃.

If R¹ is a substituted alkyl group, one or more aryl substituents, i.e. aryl groups, preferably one aryl group, are preferred.

As used herein, the term “aryl group” denotes an aromatic group which may or may not contain heteroatoms like nitrogen, oxygen or sulphur. Aryl groups which do not contain heteroatoms are preferred. Preferred aryl groups comprise up to 20 carbon atoms, more preferably up to 12 carbon atoms, for example, 10 or 6 carbon atoms. Preferred examples of aryl groups are phenyl and naphthyl, especially phenyl. Further, the aryl group may optionally be substituted by one or more, more preferably one or two, substituents. Preferably, the aryl group is substituted at the meta or para position, most preferably the para position. Suitable substituents include halo alkyl, e.g. trifluoromethyl, alkoxy, preferably alkoxy groups containing 1 to 6 carbon atoms, halo, e.g. iodo, bromo, chloro or fluoro, preferably chloro and fluoro, nitro and C₁₋₆ alkyl, preferably C₁₋₄ alkyl. Preferred C₁₋₆ alkyl groups include methyl, isopropyl and t-butyl, particularly methyl. Particularly preferred aryl substituents are chloro and nitro. However, still more preferably the aryl group is unsubstituted.

Preferred such R¹ groups are benzyl, 4-isopropylbenzyl, 4-methylbenzyl, 2-methylbenzyl, 3-methylbenzyl, 4-[t-butyl]benzyl, 4-[trifluoromethyl]benzyl, 4-methoxybenzyl, 3,4-[di-chloroThenzyl, 4-chlorobenzyl, 4-fluorobenzyl, 2-fluorobenzyl, 3-fluorobenzyl, 2,3,4,5,6-pentafluorobenzyl, 3-nitrobenzyl, 4-nitrobenzyl, 2-phenylethyl, 4-phenylbutyl, 3-pyridinyl-methyl, 4-diphenyl-methyl and benzyl-5-[(1-acetyloxyethoxy)-carbonyl]. More preferred such R¹ groups are benzyl, 4-isopropylbenzyl, 4-methylbenzyl 4-nitrobenzyl and 4-chlorobenzyl. Most preferred is benzyl.

If R¹ is a substituted alkyl group, one or more oxo substituents are preferred.

Preferably, such groups are straight-chained C₄₋₁₂ alkyl groups which are substituted by one, two or three oxo groups. Examples of such groups include 3,6-dioxa-1-octyl and 3,6,9-trioxa-1-decyl.

If R¹ is an unsubstituted alkyl group, R¹ groups that are saturated straight-chained or branched alkyl groups are preferred. If R¹ is a saturated straight-chained alkyl group, C₁₋₁₀ straight-chained alkyl group are preferred. Representative examples of suitable straight-chained alkyl groups include methyl, ethyl, n-propyl, n-butyl, n-pentyl, n-hexyl and n-octyl. Particularly preferred are C₁₋₆ straight-chained alkyl groups, most particularly preferred are C₃-C₆ straight-chained alkyl groups, e.g. n-hexyl. If R¹ is a saturated branched alkyl group, such branched alkyl groups preferably consists of a stem of 4 to 8, preferably 5 to 8 straight-chained carbon atoms which is branched by one or more C₁₋₆ alkyl groups, preferably C₁₋₂ alkyl groups. Examples of such saturated branched alkyl groups include 2-methylpentyl, 4-methylpentyl, 1-ethylbutyl and 3,3-dimethyl-1-butyl.

In compounds of formula I, each R² independently represents a hydrogen atom or a group R¹. Particularly preferred for use in the invention are those compounds of formula I in which at least one R² represents a hydrogen atom. In especially preferred compounds each R² represents a hydrogen atom.

The most preferred photosensitisers to be used in the enema preparation according to the present invention are compounds of formula I and pharmaceutically acceptable salts thereof, wherein R¹ is hexyl, more preferably n-hexyl and both R² represent hydrogen, i.e. 5-ALA hexyl ester and pharmaceutically acceptable salts thereof, preferably the HCl salts. The most preferred photosensitiser is 5-ALA hexyl ester in the form of its HCl salt.

The photosensitisers for use in the invention may be prepared by any conventional procedure available in the art, e.g. as described in WO 02/10120 to Photocure ASA. For example, esters of 5-ALA may be prepared by reaction of 5-ALA with the appropriate alcohol in the presence of base. Alternatively compounds for use in the invention may be available commercially, e.g. from Photocure ASA, Norway.

The compounds for use according to the invention may be in the form of a free amine, e.g. —NH₂, —NHR² or —NR²R², or preferably in the form of a physiologically acceptable salt. Such salts preferably are acid addition salts with physiologically acceptable organic or inorganic acids. Suitable acids include, for example, hydrochloric, nitric, hydrobromic, phosphoric, sulphuric, sulphonic and sulphonic acid derivatives. Particularly preferred salts are acid addition salts with sulphonic acid or sulphonic acid derivatives as described in WO 2005/092838 to Photocure ASA, the entire contents of which are incorporated herein by reference. Procedures for salt formation are conventional in the art.

In a further aspect the invention provides an enema preparation which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents.

An enema preparation as herein defined for use in medicine forms a yet further aspect of the invention.

The enema preparation may take any form which is suitable for administration intra-colonically which may include solution, suspension, sol and gel forms. The enemas herein described may take the form of a liquid or foam. Another aspect of the present invention thus relates to foam enemas comprising a photosensitising agent as herein described. Typical compositions of foam enemas are generally described in the prior art, see for example U.S. Pat. No. 6,432,967. Thus the carrier vehicle may also comprise an effective amount of a foaming agent such as n-butane, propane or iso-butane. Such formulations can be delivered from a pressurised container so that this is delivered to the colon as a foam which inhibits release from the target site.

In addition to 5-ALA or a precursor or derivative thereof, the enema preparations according to and for use in the invention will comprise at least one liquid pharmaceutically acceptable carrier and optionally various excipients. The liquid may be water or a physiologically acceptable solvent or a mixture of water and one or more physiologically acceptable solvents. Typical such solvents include, for example, glycerol, ethylene glycol, propylene glycol, polyethylene glycol and polypropylene glycol. A particularly preferred liquid carrier is water.

In another embodiment, oils are used as a solvent, e.g. natural and/or synthetic oils that are commonly used in pharmaceutical preparations. If oils are used, it is preferred to use a lipophilic salt of 5-ALA or a lipophilic salt and/or ester of 5-ALA, e.g. a mesylate or tosylate salt of 5-ALA or such a salt of a 5-ALA ester comprising an alkyl residue of 2-10 carbon atoms, such as hexyl or benzyl.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

Other known excipients such as buffers, preservatives, pH adjusters, etc. may be included.

The photosensitisers herein described may be used for the manufacture of an enema preparation in any conventional manner. The desired concentration of photosensitiser in the enema preparations of the invention will vary depending on several factors including the nature of the compound, the nature and form of the product in which this is presented, the nature of the cancer to be treated or diagnosed and the subject to be treated. Generally, however, the concentration of photosensitiser is conveniently in the range 0.001 to 10 mmol per litre, preferably 0.01 to 5 mmol per litre, most preferably from 0.05 to 4 mmol per litre. Particularly preferably, the photosensitiser will be used in a concentration of less than 2.5 mmol per litre.

The enema preparations according to the invention further comprise one or more of the following:
a) one or more viscosity enhancing agents;
b) one or more mucoadhesive agents or one or more mucolytic agents;
c) one or more penetration enhancers; and
d) one or more chelating agents.

The term “one or more of the following” means that the enema preparation according to the invention at least comprises one compound of the group of compounds a) to d), i.e. either a) or b) or c) or d). Alternatively, the enema preparation may comprise more than one compound of the group of compounds a) to d), e.g. one or more viscosity enhancers a) and one or more penetration enhancers c), or e.g. one or more mucolytic agents b), one or more chelating agents d) and one or more viscosity enhancing agents a).

Preferred embodiments of the enema preparation according to the invention are: an enema preparation which comprises a) one or more viscosity enhancing agents, or b) one or more mucoadhesive agents or one or more mucolytic agents, or c) one or more penetration enhancers, or d) one or more chelating agents; an enema preparation which comprises b) one or more mucolytic agents, preferably in combination with c) one or more penetration enhancers, and/or d) one or more chelating agents; an enema preparation which comprises b) one or more mucoadhesive agents, preferably in combination with c) one or more penetration enhancers, and/or d) one or more chelating agents; and an enema preparation which comprises a) one or more viscosity agents, preferably in combination with b) one or more mucoadhesive agents or mucolytic agents, and c) one or more penetration enhancers and/or d) one or more chelating agents.

The enema preparations according to and for use in the invention provide an essentially homogeneous filling of the entire colon following administration and optionally any movement of the patient. Homogeneous filling of the colon may be achieved by using a) one or more a viscosity enhancing agents. The one or more viscosity enhancing agents can be any viscosity enhancing agent used in pharmaceutical formulations. Typical viscosity enhancing agents to be used in an enema preparation according to the present invention include, for example, gelatine, tragacanth gums, xanthan gums, pectin, polysaccharides and cellulose derivatives like carboxymethyl cellulose, methyl cellulose, hydroxypropyl cellulose, etc.

One preferred aspect of the present invention relates to enema preparations that change viscosity over time: the viscosity is low during administration but increases after the enema is instilled into the area of interest. This can be achieved by administration of enema preparations comprising one or more viscosity agents which comprise swellable compounds, typically polysaccharides, where the swellable compounds are not fully swollen before administration of the enema preparation. Alternatively, one or more viscosity agents may be used which increase the viscosity of the liquid when warmed up from around room temperature to body temperature. Several such viscosity agents are generally known in the art of galenic formulations.

The enema preparations according to the invention may comprise b) one or more mucoadhesive agents. Mucoadhesive agents in the enema preparation of the invention help to improve adhesion to the colon wall and thus achieve uniform coating of the target site. As used herein, “mucoadhesive agent” refers to any agent which exhibits an affinity for a mucosa surface, i.e. which adheres to that surface through the formation of bonds which are generally non-covalent in nature, whether binding occurs through interaction with the mucous or the underlying cells. The mucoadhesive agent can be any mucoadhesive agent used in pharmaceutical formulations. Typical mucoadhesive agents to be used in the current enema formulations include those described in WO 02/09690, the entire contents of which are incorporated herein by reference.

Mucoadhesive agents which may be used in the enema preparations of the invention may be natural or synthetic, polyanionic, polycationic or neutral, water-soluble or water-insoluble, but are preferably large, more preferably having a molecular weight of 500 to 3000 kDa, e.g. 1000 to 2000 kDa, water-insoluble cross-linked, e.g. containing 0.05 to 2%, e.g. 0.75 to 1.5% cross-linker by weight of the total polymer, prior to any hydration, water-swellable polymers capable of forming hydrogen bonds. Preferably mucoadhesives according to the invention have a mucoadhesive force greater than 100, especially preferably greater than 120, particularly greater than 150, as assessed according to the method of Smart et al., 1984, J. Pharm. Pharmacol., 36, p 295-299, expressed as a percent relative to a standard in vitro.

Appropriate mucoadhesive agents include, but are not limited to poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters. Alternatively, cellulose derivatives may be used such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof. Other naturally occurring or synthetic polymers may also be used such as gums, e.g. xanthan gum, guar gum, locust bean gum, tragacanth gums, karaya gum, ghatti gum, cholla gum, psillium seed gum and gum arabic; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; lipophilic formulations containing polysaccharides, e.g. Orabase (Bristol Myers Squibb); carbohydrates such as polysubstituted with groups such as sulphate, phosphate, sulphonate or phosphonate, e.g. sucrose octasulphate; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as polyvinyl alcohols, polyvinylmethyl ethers, polyvinylpyrrolidone, polycarboxylated vinyl polymers such as polyacrylic acid as mentioned above; polysiloxanes; polyethers; polyethylene oxides and glycols; polyalkoxys and polyacrylamides and derivatives and salts thereof.

The above described polymeric mucoadhesive agent may also be cross-linked and may be in the form of copolymers. Preferably poly(acrylic acid) polymers or copolymers, e.g. with di- or poly functional allyl ethers or acrylates to make the polymer insoluble, which have preferably been cross-linked, e.g. using a polyalkenyl polyether, are employed which have a high molecular weight and are thixotropic. Appropriate mucoadhesive agents having this form are available commercially (e.g. from Goodrich) as polycarbophil, e.g. Noveon AA-1, Carbomer (Carbopol), e.g. Carbopol EX165, EX214, 434, 910, 934, 934P, 940, 941, 951, 974P and 1342.

Some of the preferred mucoadhesive agents for use in the enema preparations of the invention include, polyacrylic hydrogels, chitosan, polyvinyl alcohol, hydroxypropyl cellulose, hydroxypropyl methyl cellulose, sodium alginate, scleroglucan, xanthan gum, pectin, orabase and polygalactonic acid.

It will be appreciated from the discussion herein that the viscosity enhancing agent and/or the mucoadhesive agent may itself comprise the carrier or excipient and that in such cases a further carrier or excipient is optionally present. Some of the one or more compounds a) and b) impact on and prolong the release of the active photosensitising agent. Such components are well known in the art and may include, for example, guar gum or other gums. The desired content of such components, e.g. gums, in the formulation can readily be determined by those skilled in the art and may, for example, be in the range 1 to 10 weight-%.

The enema preparation according to the invention may comprise b) one or more mucolytic agents. Such agents facilitate the removal of mucus in the colon by destroying or dissolving mucin and thus facilitate the uptake of the photosensitiser into the tissue. The use of such enemas thus shortens the time of the enema procedure: the amount of photosensitiser which is taken up into the colon tissue per time unit is higher if the tissue is not coated (or to a lesser degree coated) by mucus.

Suitable mucolytic agents are compounds with a free —SH group, preferably cysteamine, pepsin and N-acetyl-1-cysteine or pharmaceutically acceptable salts thereof. When present, the one or more mucolytic agents may conveniently be used at a concentration of 0.5 to 10% by weight based on the enema preparation in which it is present.

In an alternative embodiment, mucolytic agents are comprised in the products which are used to evacuate the lower part of the gastrointestinal system prior to the instillation of the enema preparation. Hence laxatives comprising mucolytic agents or cleansing enemas comprising mucolytic agents are preferably used followed by instillation of an enema preparation according to the invention.

The enema preparations according to the invention may comprise c) one or more penetration enhancers which have a beneficial effect in enhancing the photosensitising effect of the photosensitiser by enhancing the penetration of said photosensitiser in tissues. Such enema preparations require less incubation time since more photosensitiser is taken up into the tissue in one time unit compared to enemas without penetration enhancers. Preferred enema preparations according to and for use in the invention are thus enema preparations which comprise c) one or more penetration enhancers. Suitable penetration enhancers include, in particular, dialkylsulphoxides such as dimethylsulphoxide (DMSO). The penetration enhancer may be any of the skin penetration assisting agents described in the pharmaceutical literature, e.g. chelators like EDTA, surfactants such as sodium dodecyl sulphate, non-surfactants, bile salts like sodium deoxycholate and fatty acids, e.g. oleic acid. Examples of appropriate penetration enhancers include isopropanol, HPE-101 (available from Hisamitsu), DMSO and other dialkylsulphoxides, in particular n-decylmethyl sulphoxide (NDMS), dimethylsulphacetamide, dimethylformamide (DMFA), dimethylacetamide, glycols, glycolic acid, various pyrrolidone derivatives (see Woodford et al., J. Toxicol. Cut. & Ocular Toxicology, 1986, 5: 167-177) and Azone® (see Stoughton et al., Drug Dpv. Ind. Pharm. 1983, 9: 725-744) or mixtures thereof. Preferred penetration enhancers are EDTA, glycolic acid and DMSO.

The penetration enhancer may conveniently be provided in a concentration range of 0.2 to 50% by weight of the total weight of the enema preparation in which it is present, e.g. in an amount of about 10% by weight.

The enema preparations according to the invention may comprise d) one or more chelating agents which have a beneficial effect in enhancing the accumulation of protoporphyrin (Pp) since the chelation of iron by the chelating agent prevents its incorporation into Pp to form haem by the action of the enzyme ferrochelatase, thereby leading to a build up of Pp. The photosensitising effect is therefore enhanced. Enema preparations which include one or more chelating agents are thus particularly preferred for use in the invention since their use shortens the time of the enema procedure: less photosensitiser needs to be taken up into the tissue in one time unit to achieve a similar fluorescence compared to enemas without chelating agents. Alternatively, less amount of photosensitiser may be used in the enema preparation.

Suitable chelating agents that may be included in the enema preparations of the invention include aminopolycarboxylic acids, such as any of the chelants described in the literature for metal detoxification or for the chelation of paramagnetic metal ions in magnetic resonance imaging contrast agents. Particular mention may be made of EDTA, CDTA (cyclohexane triamine tetraacetic acid), DTPA and DOTA and well known derivatives and analogues thereof. EDTA and DTPA are particularly preferred. To achieve the iron-chelating effect, desferrioxamine and other siderophores may also be used, e.g. in conjunction with aminopolycarboxylic acid chelating agents such as EDTA.

Where present, the one or more chelating agents may conveniently be used at a concentration of 0.05 to 20%, e.g. 0.1 to 10% by weight based on the enema preparation in which it is present.

The enema preparations of the invention may additionally comprise an anti-cancer agent. Thus viewed from a further aspect the invention provides an enema preparation comprising a photosensitiser which is 5-ALA or a precursor or derivative thereof, e.g. a 5-ALA ester, together with an anti-cancer agent for use in the treatment of cancer. A preferred embodiment of such an enema preparation is an enema preparation comprising a photosensitiser which is 5-ALA or a precursor or derivative thereof, e.g. a 5-ALA ester, together with an anti-cancer agent and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents.

Viewed from a still further aspect the invention provides a kit or pack containing an enema preparation as hereinbefore defined, and separately an anti-cancer agent for simultaneous, separate or sequential use in a method of treating cancer.

Preferred anti-cancer agents present in the pharmaceutical product and kit of the invention are anti-neoplastic agents. Representative examples of anti-neoplastic agents include alkaloids, e.g. vincristine, vinblastine, vinorelbine, topotecan, teniposiode, paclitaxel, etoposide and docetaxel, alkylating agents, e.g. alkyl sulfonates such as busulfan, aziridines, e.g. carboquone, ethylenimines and methylmelamines, nitrogen mustards, e.g. chlorambucil, cyclophosphamide, estramustin, ifosfamide and melphalan, nitrosurea derivatives, e.g. carmustine and lomustine, antibiotics, e.g. mitomycins, doxorubicin, daunorubicin, epirubicin and bleomycins, antimetabolites, e.g. folic acid analogues and antagonists such as methotrexate and raltitrexed, purine analogues, e.g. 6-mercaptopurine, pyrimidine analogues, e.g. tegafur, gemcitabine, fluorouracil and cytarabine, cytokines, enzymes such as L-asparginase, ranpirnase, immunomodulators, e.g. interferons, immunotoxins, monoclonal antibodies, taxanes, topoisomerase inhibitors, platinum complexes like carboplatin, oxaliplatin and cisplatin and hormonal agents such as androgens, estrogens, antiestrogens and aromatase inhibitors. Other anti-neoplastic agents for use in the invention include imiquimod, irenotecan, leucovorin, levamisole, etoposide and hydroxyurea.

Particularly preferred anti-cancer agents for use in the invention include 5-fluorouracil, imiquimod, cytokines, mitomycin C, epirubicin, irenotecan, oxalipatin, leucovorin, levamisole, doxorubicin, cisplatin, etoposide, doxirubicin, methotrexate, taxanes, topoisomerase inhibitors, hydroroxyurea and vinorelbine. Yet more preferred for use as anti-cancer agents are antibiotics such as mitomycin and pyrimidine analogues such as 5-fluorouracil.

The enema preparations of the invention may additionally comprise one or more non-photosensitising agents. Such agents may, for example, include antibiotics for treatment of various bacterial infections, anti-inflammatory agents like 5-aminosalicylic acid and derivatives thereof for the treatment of inflammatory bowel diseases and inflammatory conditions in the lower gastrointestinal tract, or other drugs such as 5-HT ligands and steroids.

Viewed from a further aspect the invention thus provides an enema preparation comprising a photosensitiser which is 5-ALA or a precursor or derivative thereof, e.g. a 5-ALA ester, together with a non-photosensitising agent. A preferred embodiment of such an enema preparation is an enema preparation comprising a photosensitiser which is 5-ALA or a precursor or derivative thereof, e.g. a 5-ALA ester, together with a non-photosensitising agent and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents.

In the case of anti-inflammatory agents, such agents may also be used orally in a period before the enema procedure and/or may be present in the products which are used to evacuate the lower part of the gastrointestinal system prior to the instillation of the enema preparation. Hence the use of oral anti-inflammatory agents and/or laxatives or cleansing enemas comprising anti-inflammatory agents is preferably followed by instillation of an enema preparation according to the invention which comprises a photosensitiser which is 5-ALA or a precursor or derivative thereof, e.g. an ALA ester, and at least one pharmaceutically acceptable carrier or excipient and which further comprises one or more of the following:
a) one or more viscosity enhancing agents;
b) optionally one or more mucolytic agents;
c) one or more penetration enhancers; and
d) one or more chelating agents.

The use of anti-inflammatory agents may be beneficial to help to reduce unspecific fluorescence of inflammatory lesions which may lead to “false-positive” results in the PDD procedure.

Viewed from a still further aspect the invention provides a kit or pack containing an enema preparation as hereinbefore defined, and separately a non-photosensitising agent for simultaneous, separate or sequential use in a method of treating a non-cancerous condition.

Diagnostic agents may also be present in the preparations herein described or, alternatively, may be administered in combination with the enema preparations. Another aspect of the present invention thus relates to an enema preparation comprising 5-ALA or a precursor or derivative thereof and a diagnostic agent, for example an X-ray contrast agent or an MRI contrast agent. A preferred embodiment of such an enema preparation is an enema preparation comprising a photosensitiser which is 5-ALA or a precursor or derivative thereof, e.g. a 5-ALA ester together with a diagnostic agent and at least one pharmaceutically acceptable carrier or excipient and which further comprises one or more of the following:
a) one or more viscosity enhancing agents;
b) one or more mucoadhesive agents or one or more mucolytic agents;
c) one or more penetration enhancers; and
d) one or more chelating agents.

Viewed from a still further aspect the invention provides a kit or pack containing an enema preparation as hereinbefore defined, and separately a diagnostic agent for instance an X-ray contrast agent or an MRI contrast agent, for simultaneous, separate or sequential use in a method of diagnosis or as a follow-up to treatment of cancer or a non-cancerous condition.

The preferred X-ray contrast agents to be used according to the present invention are barium sulphate and non-ionic X-ray contrast agents like for example iohexyl, iopamoidol and iodixanol. The enema formulations comprising an X-ray contrast agent according to the present invention comprise typically 2-30 weight-% of the X-ray contrast agent in addition to the photosensitising agent. Suitable MRI contrast agents are those based on iron, manganese or gadolinium like gadopentetate. When used in combination with an X-ray contrast agent or an MRI contrast agent, the enema preparations herein described are able to provide double contrast enhancement, i.e. PDD plus X-ray or PDD plus MRI. Alternatively, the contrast agent might be present in the formulation to visually check in X-ray imaging or MRI that the enema is present in the whole colon or at least present at the site or area of interest.

The enema preparations according to the invention may be administered in combination with a second photosensitising agent, preferably one comprising 5-ALA or a precursor or derivative thereof. Typically, the second agent will be administered by an alternative mode of administration, e.g. orally.

Viewed from a still further aspect the invention provides a kit or pack containing a pharmaceutical product as hereinbefore defined, and separately an oral composition comprising a second photosensitiser which comprises 5-ALA or a precursor or derivative thereof. The oral composition is preferably an oral composition intended for PDD or PDT of the lower part of the gastrointestinal system. Such compositions are typically solid formulations like tablets, pellets, capsules containing non-aqueous formulations. Suitable formulations include those described in WO 2009/074811.

The enema preparations according to the invention may be provided in “ready-to-use” form. Alternatively, these may be provided in a kit or pack comprising one or more separate components, e.g. two components which when mixed together provide the desired preparation. Another preferred aspect of the present invention relates to enemas comprising two components that are mixed before use. This two-component system typically comprises two vials; one vial contains a preparation comprising 5-ALA or a precursor or derivative thereof which preferably will be formulated as a solid, optionally with other solid materials; and the second vial contains a liquid. The solid material is dissolved in the liquid phase immediately prior to use.

“Ready-to-use” enemas will generally be provided in a “single-use” sealed disposable container of plastic or glass. Those formed of a polymeric material should have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

Prior to administration of the enema preparation of the invention it is usual to first cleanse the colonic area. This may be achieved using a second enema intended for cleansing purposes. Viewed from a still further aspect the invention provides a kit or pack containing an enema preparation as hereinbefore defined, and separately a second enema for cleansing. This second enema may be any commercially available cleansing enema.

Any of the kits or packs herein described may further optionally comprise a balloon intended for use in preventing leakage of the enema, especially that containing the photosensitising agent, following administration.

The enema preparation of the invention can be administered by known intra-colonic methods. For example, when provided in a flexible container this can be administered to a patient by squeezing the container; this can be done by the patient or by a nurse or other medical assistant. Another option is to administer the enema based on gravity forces by placing the enema above the patient or the enema might be administered using various apparatus available in the clinic or at the doctor's office. Such apparatus are for example described in U.S. Pat. No. 4,504,270, U.S. Pat. No. 4,419,099 and U.S. Pat. No. 4,117,847. The amount of the enema preparation administered will be selected according to its use, the age, sex and other conditions of the patient, and the severity of the condition. Typically the total volume of the enema will vary from 30 ml to 1500 ml. A typical enema volume for diagnosis or therapy of, for example, colorectal cancer is around 500 ml.

After administration of the enema preparation according to the invention containing the photosensitiser, the site to be treated or diagnosed is exposed to light to achieve the desired photosensitizing effect. The length of time following administration at which the light exposure takes place will depend on the nature of the enema e.g. whether this is in liquid or foam form, whether this contains any delayed release agents, etc., the condition to be treated or diagnosed, etc. Generally, it is necessary that the photosensitiser should reach an effective tissue concentration at the site of the condition (e.g. cancer) prior to photoactivation. This can generally take in the region of from 0.5 to 24 hours, preferably 0.5 to 3 hours.

In a preferred treatment or diagnosis procedure, the photosensitiser is applied to the affected site followed by irradiation e.g. after a period of about 0.5 to 3 hours). If necessary, e.g. during treatment, this procedure may be repeated, e.g. up to a further 3 times, at intervals of up to 30 days, e.g. 7-30 days. In those cases where this procedure does not lead to a satisfactory reduction in, or complete healing of, the condition e.g. cancer, an additional treatment may be performed several months later.

For therapeutic purposes, methods for irradiation of different areas of the body, e.g. by lamps or lasers are well known in the art (see for example Van den Bergh, Chemistry in Britain, May 1986 p. 430-439). The wavelength of light used for irradiation may be selected to achieve an efficacious photosensitizing effect. The most effective light is light in the wavelength range 300-800 nm, typically 400-700 nm where the penetration of the light is found to be relatively deep. The irradiation will in general be applied at a dose level of 10 to 100 Joules/cm² with an intensity of 20-200 mW/cm² when a laser is used or a dose of 10-100 J/cm² with an intensity of 50-150 mW/cm² when a lamp is applied. For treatment, irradiation is preferably performed for 5 to 30 minutes, preferably for 15 minutes. For diagnosis, irradiation is preferably performed during the whole diagnostic procedure or during a part thereof, e.g., when combined with white light detection. A single irradiation may be used or alternatively a light split dose in which the light dose is delivered in a number of fractions, e.g. a few minutes to a few hours between irradiations, may be used. Multiple irradiations may also be applied. Devices specifically adapted for use in irradiating the colonic area will preferably be used, e.g. an endoscope.

For diagnostic use, the area is preferably first inspected using white light. Suspicious areas are then exposed to blue light (typically ranging from 400-450 nm). The emitted fluorescence (635 nm) is then used to selectively detect affected cancerous or non-cancerous tissues having a higher metabolic activity than healthy tissue. When carrying out diagnosis, it is preferable to use blue light using a device e.g. an endoscope and assessing the fluorescence.

An advantage of the enemas according to the present invention relates to their efficacy, sensitivity and specificity when used for diagnostic purposes and the overall therapeutic outcome for PDT. In addition, the present enemas have a high degree of patient compliance. The enemas of the present invention are also remarkably stable. For example, the pharmaceutical products can be stored, e.g. at room temperature and humidity, for at least 12 months, more preferably at least 24 months, still more preferably at least 36 months or more.

The products and methods of the invention may be used to treat and/or diagnose cancer or non-cancerous conditions in the lower gastrointestinal tract, in particular in the large intestine (colon), especially in the sigmoid colon, the descending colon and the rectum. Such conditions include inflammatory bowel diseases, colorectal cancer, ulcerative colitis, Crohn's disease, irritable bowel disease, etc. Inflammatory bowel diseases are inflammatory diseases of the large and small intestines which may be caused by a number of factors. In most patients the regions affected extend over a wide range of the colon, e.g. to the descending colon or transverse colon. Use of the enema preparations herein described ensures that the desired therapeutic or diagnostic effects are achieved because the active ingredients can directly reach the affected regions.

The invention will now be described in more detail by way of the following non-limiting examples:

### EXAMPLE 1

### Two-Component Enema Comprising 5-ALA Hexyl Ester for Therapeutic Treatment of Colorectal Cancer

### Component 1:

| | |
|---|---|
| 5-ALA hexyl ester HCl | 1000 mg (equivalent to 850 mg |
| | 5-ALA hexyl ester) |
| Hydroxyethyl cellulose | 1000 mg |

The components are filled into a plastic container (750 ml).

### Component 2:

| | | |
|---|---|---|
| Glycerol | 10 | grams |
| Sodium laurylsulphate | 200 | mg |
| Purified water | ad. 500 | ml |

The components are filled into a plastic container (500 ml).

Prior to use the components are mixed and used as follows:

Component 1 is preheated in a water bath to 45-50° C. Component 1 is added to the container with component 2. The container is shaken for 3 minutes and allowed to reach body temperature before the solution is administered as an enema. After administration of the enema, the patient is moved from side to side and also with head about 20 degrees up and down for 10 minutes. The viscosity of the enema is at this time higher than the viscosity of the enema during administration.

The enema is removed after 30 min to 1 hour and the rectum/colon examined with PDD followed by PDT.

### EXAMPLE 2

### Two-Component Kit Comprising an Enema and Tablets for Oral Administration for Treatment of Colorectal Cancer

### Coated Tablets Comprising 5-ALA Hexyl Ester HCl (Kit Component 1):

| | |
|---|---|
| Microcrystalline cellulose (Avicel PH-102) | 380 mg |
| Lactose monohydrate | 340 mg |
| 5-ALA hexyl ester HCl | 150 mg |
| Magnesium stearate | 10 mg |

The components are mixed and tablets are prepared by direct compression. Tablet diameter: 13 mm. The tablets are coated with an acetone solution of Eudragit S-100 (6%) and triethyl citrate (1%) and dried.

### Enema Comprising 5-ALA Hexyl Ester HCl (Kit Component 2):

### Enema Component A:

| | |
|---|---|
| 5-ALA hexyl ester HCl | 500 mg (equivalent to 425 mg |
| | 5-ALA hexyl ester) |
| Sodium chloride | 4 grams |

The components are filled into a plastic container (750 ml).

### Enema Component B:

| | | |
|---|---|---|
| Glycerol | 30 | grams |
| Sodium laurylsulphate | 100 | mg |
| Aqueous buffer pH 6.0 | ad. 500 | ml |

The components are filled into a plastic container (500 ml).

The components of the kit are used as follows:

Two tablets are administered 20 hours before PDT.

Enema component B is added to enema component A. The mixture is shaken for 30 seconds. The enema is administered to the patient 1 hour before PDT by being instilled over a period of 30 min following a 15-30 min rest. The patient is moved as described in Example 1 to secure good filling of the colon. The enema is then removed and PDT is performed.

### EXAMPLE 3

### Enema Comprising 5-ALA Hexyl Ester in Iohexyl Solution (Two Component)

### Component 1:

| | |
|---|---|
| 5-ALA hexyl ester HCl | 300 mg (257 mg 5-ALA hexyl ester) |

### Component 2:

| | | |
|---|---|---|
| Iohexol | 43.3 | grams |
| Trometamol | 180 | mg |
| EDTA calcium | 15 | mg |
| Hydrochloric acid to adjust pH to 6.8-7.0 | | |
| Purified water | ad. 150 | ml |

Prior to use the components are mixed and used as follows:

Enema component 2 is added to enema component 1. The mixture is shaken for 30 seconds. The enema is administered to the patient 2 hours before PDT. The patient is moved as described in Example 1 to secure good filling of the colon. The filling of the colon is followed by X-ray imaging. The enema is removed and PDT is performed.

### EXAMPLE 4

### Two-Component Enema Comprising 5-ALA Benzyl Ester for Therapeutic Treatment of Colorectal Cancer

### Component 1:

| | |
|---|---|
| 5-ALA benzyl ester HCl | 1028 mg (equivalent to 884 mg |
| | 5-ALA benzyl ester) |
| Hydroxyethyl cellulose | 500 mg |
| EDTA trisodium | 10 mg |

The components are filled into a plastic container (750 ml).

### Component 2:

| | | |
|---|---|---|
| Glycerol | 10 | grams |
| Stearic acid | 0.5 | gram |
| Chitosan lactate (FMC biopolymer) | 1.0 | g |
| Isopropanol | 20 | grams |
| Purified water | ad. 500 | ml |

The components are filled into a plastic container (500 ml).

Prior to use the components are mixed and used as follows:

Component 1 is pre-heated in a water bath to 45-50° C. Component 1 is added to the container with component 2. The container is shaken for 3 minutes and allowed to reach body temperature before the solution is administered as an enema. After administration of the enema, the patient is moved from side to side and also with head about 20 degrees up and down for 10 minutes. The viscosity of the enema is at this time higher than the viscosity of the enema during administration.

The enema is removed after 30 min to 1 hour and the rectum/colon examined with PDD followed by PDT.

### EXAMPLE 5

### Two-Component Enema Comprising 5-ALA Benzyl Ester for Diagnosis of Colorectal Cancer

### Component 1:

| | |
|---|---|
| 5-ALA benzyl ester HCl | 514 mg (equivalent to 442 mg |
| | 5-ALA benzyl ester) |
| Hydroxyethyl cellulose | 200 mg |
| EDTA trisodium | 10 mg |

The components are filled into a plastic container (750 ml).

### Component 2:

| | | |
|---|---|---|
| Glycerol | 10 | grams |
| Sodium laurylsulphate | 200 | mg |
| Pectin (GENU Type: LM-104 AS-Z) | 0.5 | g |
| Dimethyl sulphoxide | 20 | grams |
| Purified water | ad. 500 | ml |

The components are filled into a plastic container (500 ml).

Prior to use the components are mixed and used as follows:

Component 1 is pre-heated in a water bath to 45-50° C. Component 1 is added to the container with component 2. The container is shaken for 3 minutes and allowed to reach body temperature before the solution is administered as an enema. After administration of the enema, the patient is moved from side to side and also with head about 20 degrees up and down for 10 minutes. The viscosity of the enema is at this time higher than the viscosity of the enema during administration.

The enema is removed after 30 min to 1 hour and the rectum/colon examined with PDD followed by PDT.

### EXAMPLE 6

### Two-Component Enema Comprising 5-ALA Benzyl Ester for Therapeutic Treatment of Colorectal Cancer

### Component 1:

| | |
|---|---|
| 5-ALA benzyl ester HCl | 1028 mg (equivalent to 884 mg |
| | 5-ALA benzyl ester) |
| Hydroxyethyl cellulose | 500 mg |
| Acetylcysteine | 200 mg |
| EDTA trisodium | 10 mg |

The components are filled into a plastic container (750 ml).

### Component 2:

| | | |
|---|---|---|
| Glycerol | 10 | grams |
| Stearic acid | 0.5 | gram |
| Polysorbate 60 | 0.3 | grams |
| Tween 20 | 0.4 | grams |
| Polyacrylic acid (Fluka) | 1.0 | g |
| Isopropanol | 20 | grams |
| Dimethylsulphoxide | 5 | grams |
| Purified water | ad. 500 | ml |

The components are filled into a plastic container (500 ml).

Prior to use the components are mixed and used as follows:

Component 1 is pre-heated in a water bath to 45-50° C. Component 1 is added to the container with component 2. The container is shaken for 3 minutes and allowed to reach body temperature before the solution is administered as an enema. After administration of the enema, the patient is moved from side to side and also with head about 20 degrees up and down for 10 minutes. The viscosity of the enema is at this time higher than the viscosity of the enema during administration.

The enema is removed after 30 min to 1 hour and the rectum/colon examined with PDD followed by PDT.

### EXAMPLE 7

### Two-Component Enema Comprising 5-ALA Methyl Ester for Therapeutic Treatment of Colorectal Cancer

### Component 1:

| | | |
|---|---|---|
| 5-ALA methyl ester mesylate salt | 2000 | mg |
| Methyl cellulose | 100 | mg |
| Salicylic acid | 200 | mg |
| EDTA trisodium | 10 | mg |
| Chitosan | 200 | mg |

The components are filled into a plastic container (750 ml).

### Component 2:

| | | |
|---|---|---|
| Glycerol | 10 | grams |
| Stearic acid | 0.5 | gram |
| Polysorbate 20 | 0.3 | grams |
| Brij 30 | 0.2 | grams |
| Polyacrylic acid(Fluka) | 1.0 | g |
| Isopropanol | 30 | grams |
| Purified water | ad. 500 | ml |

The components are filled into a plastic container (500 ml).

Prior to use the components are mixed and used as follows:

Component 1 is pre-heated in a water bath to 45-50° C. Component 1 is added to the container with component 2. The container is shaken for 3 minutes and allowed to reach body temperature before the solution is administered as an enema. After administration of the enema, the patient is moved from side to side and also with head about 20 degrees up and down for 10 minutes. The viscosity of the enema is at this time higher than the viscosity of the enema during administration.

The enema is removed after 30 min to 1 hour and the rectum/colon examined with PDD followed by PDT.

### EXAMPLE 8

### Two-Component Kit Comprising an Enema and Tablets for Oral Administration for Treatment of Colorectal Cancer

### Coated Tablets Comprising 5-ALA Hexyl Ester HCl (Kit Component 1):

| | |
|---|---|
| Microcrystalline cellulose (Avicel PH-102) | 380 mg |
| Lactose monohydrate | 340 mg |
| 5-ALA hexyl ester HCl | 150 mg |
| Magnesium stearate | 10 mg |

The components are mixed and tablets are prepared by direct compression. Tablet diameter: 13 mm. The tablets are coated with an acetone solution of Eudragit S-100 (6%) and triethyl citrate (1%) and dried.

Enema comprising 5-ALA hexyl ester HCl (kit component 2):

### Enema Component A:

| 5-ALA hexyl ester HCl | 500 mg (equivalent to 425 mg | |
|---|---|---|
| | 5-ALA hexyl ester) | |
| Sodium chloride | 4 | grams |
| EDTA trisodium | 30 | mg |
| Hydroxyethylcellulose | 400 | mg |
| Pectin (Copenhagen pectin) | 300 | mg |
| Sodium lauryl sulphate | 300 | mg |

The components are filled into a plastic container (750 ml).

### Enema Component B:

| | |
|---|---|
| Glycerol | 30 grams |
| Ethanol | 10 grams |
| Isopropanol | 15 grams |
| Aqueous buffer pH 6.0 | ad. 500 ml |

The components are filled into a plastic container (500 ml).

The components of the kit are used as follows:

Two tablets are administered 20 hours before PDT.

Enema component B is added to enema component A. The mixture is shaken for 30 seconds. The enema is administered to the patient 1 hour before PDT by being instilled over a period of 30 min following a 15-30 min rest. The patient is moved as described in Example 1 to secure good filling of the colon. The enema is then removed and PDT is performed.

1. An enema preparation which comprises a photosensitiser which is 5-ALA or a precursor or derivative thereof, or a pharmaceutically acceptable salt thereof, and at least one pharmaceutically acceptable carrier or excipient and which further comprises one or more of the following: a) one or more viscosity enhancing agents; b) one or more mucoadhesive agents or one or more mucolytic agents; c) one or more penetration enhancers; and d) one or more chelating agents.

2. An enema preparation according to claim 1, wherein said photosensitiser is a 5-ALA derivative or a pharmaceutically acceptable salt thereof.

3. An enema preparation according to claim 1, wherein said photosensitiser is a compound of formula I or a pharmaceutically acceptable salt thereof:
R²₂N—CH₂COCH₂—CH₂CO—OR¹ (I)
wherein
R¹ represents a substituted or unsubstituted alkyl group; and
R² each independently represents a hydrogen atom or a group R¹.

4. An enema preparation according to claim 3, wherein each R² represents hydrogen and R¹ represents an unsubstituted alkyl group.

5. An enema preparation according to any preceding claim, wherein said enema preparation comprises a) one or more viscosity enhancing agents or b) one or more mucoadhesive agents or one or more mucolytic agents or c) one or more penetration enhancers or d) one or more chelating agents.

6. An enema preparation according to claim 1, wherein said enema preparation comprises b) one or more mucolytic agents.

7. An enema preparation according to claim 1, wherein said enema preparation comprises b) one or more mucoadhesive agents.

8. An enema preparation according to claim 1, wherein said enema preparation comprises a) one or more viscosity agents.

9. (canceled)

10. A method of photodynamic treatment or diagnosis of cancer or non-cancerous conditions in the lower part of the gastrointestinal tract comprising the step of administering to a patient the enema preparation of claim 1.

11. The method of claim 10 wherein the condition is a non-cancerous condition in the lower part of the gastrointestinal tract.

12. The method of claim 10, further comprising the steps of: (i) optionally waiting for a time period for the photosensitiser to achieve an effective tissue concentration at the desired site; and (ii) photoactivating the photosensitiser.

13. The method of claim 12, wherein prior to the step of administering to a patient the enema preparation, the lower part of the gastrointestinal system of said patient is evacuated.

14. An enema preparation according to claim 1, which further comprises an anti-cancer agent or a non-photosensitising agent.

15. A kit or pack containing the enema preparation of claim 1, and separately an oral composition comprising a second photosensitiser which is 5-ALA or a precursor or derivative thereof, or a pharmaceutically acceptable salt thereof.

16. An enema preparation according to claim 2, wherein the photosensitiser is a 5-ALA ester or a pharmaceutically acceptable salt thereof.

17. An enema preparation according to claim 4, wherein R¹ represents a C₁-C₆ alkyl group.

18. An enema preparation according to claim 6, further comprising one or more penetration enhancers and/or one or more chelating agents.

19. An enema preparation according to claim 7, further comprising c) one or more penetration enhancers and/or d) one or more chelating agents

20. An enema preparation according to claim 8, further comprising b) one or more mucoadhesive agents or mucolytic agents, and
c) one or more penetration enhancers and/or
d) one or more chelating agents

21. The method of claim 10 wherein the cancer or non-cancerous condition is in the colon or rectum.

22. The method of claim 10 wherein the condition is selected from inflammatory bowel disease, ulcerative colitis, Crohn's disease and irritable bowel syndrome.

23. The method of claim 13, wherein the lower part of the gastrointestinal system of said patient is evacuated by using a cleansing enema or a laxative.

24. An enema preparation according to claim 1, which further comprises one or more of an antibiotic, an X-ray contrast agent and an MRI contrast agent.
